(19) United States
(12) Patent Application Publication
(10) Pub. No.: US 2016/0097025 A1
(43) Pub. Date: Apr. 7, 2016

(54) PHOTOSYNTHETIC MICROORGANISM CULTURE APPARATUS, LIGHTING DEVICE, AND PHOTOSYNTHETIC MICROORGANISM CULTURE METHOD

(71) Applicants: Kabushiki Kaisha Koshino Gijutsu Kenkyusho, Osaka (JP); Kabushiki Kaisha Koshino Nichiei Agri Institute, Osaka (JP); Kabushiki Kaisha So-laku, Tokyo (JP); Shintaro Koshino, Osaka (JP); Yosuke Hasegawa, Tokyo (JP)

(72) Inventors: Yosuke Hasegawa, Tokyo (JP); Mitsuyoshi Tsukahara, Tokyo (JP); Shintaro Koshino, Osaka (JP)

(21) Appl. No.: 14/920,155

(22) Filed: Oct. 22, 2015

(30) Foreign Application Priority Data
May 19, 2015 (JP) 2015-102230

(51) Int. Cl. C12M 1/00

(52) U.S. Cl. CPC C12M 21/02; C12M 31/08; C12M 41/08; C12M 31/10

(57) ABSTRACT

A photosynthetic microorganism culture apparatus includes: a fermenter in which a culture solution including a photosynthetic microorganism is stored; a light incidence region through which light enters the culture solution being formed in the fermenter; one or a plurality of light sources that emit collimated light; a reflection mechanism that reflects the collimated light emitted from the light source in a predetermined direction as reflected collimated light; and a controller that causes the reflected collimated light reflected by the reflection mechanism to travel periodically in a previously-fixed direction to irradiate the light incidence region with the reflected collimated light.

This application claims the priority benefit of Patent Application No. 2015-102230 filed in Japan on May 19, 2015.

### BACKGROUND OF THE INVENTION

1. Field of the Invention

The present invention relates to, for example, a photosynthetic microorganism culture apparatus that uses an artificial light source to culture a photosynthetic microorganism such as a microalga.

2. Description of the Related Art

Conventionally, microaglae such as chlorella and spirulina are artificially cultured. Generally microaglae are cultured by sunlight in an artificial pool located outdoors. Relying upon sunlight, microaglae can be cultivated outdoors at low cost. However, contamination from foreign substances can occur during a harvest. So, for hygenics, a microalgae fermenter should be located indoors.

In the case that the fermenter is provided indoors, sunlight is guided to the fermenter through an optical fiber, or artificial lights such as an LED light source are used (for example, see JP 2011-250760 A). The fermenter is made transparent so that the amount of the culture solution that receives light is maximized with respect to restricted amount of sunlight that is available indoors (for example, see JP 2014-117273 A).

When the photosynthetic microorganism is cultured outdoors, space efficiency or energy efficiency rarely is a problem. But when the photosynthetic microorganism is cultured indoors, it is generally necessary to improve the space efficiency. When the fermenter is irradiated with light from above, the fermenter is formed into a vertically tall shape, reducing the floor space required. However, when the photosynthetic microorganism proliferates in the fermenter and the density of the photosynthetic microorganism in the culture solution increases, some of the irradiating light is blocked by the microorganism near the liquid level. Less light reaches the bottom of the vertically tall fermenter, and the amount of cultivable microorganism is limited with respect to a volume of the fermenter. JP 2014-117273 A discloses a fermenter that has a transparent sidewall. When the fermenter has a transparent sidewall, the culture solution in the fermenter can be irradiated with the light not only from above but also from the side.

However, even if the sidewall of the fermenter is made transparent, light reaching the fermenter through the sidewall of the fermenter is still blocked by the microorganism when the microorganism density increases in the culture solution. As a result, little light reaches a central portion of the fermenter. Consequently, when lateral irradiation is used, a plurality of light sources are sometimes installed around the fermenter. More space is required to install light sources around the fermenter. As a result, the improvement in space efficiency is limited.

The amount of emitted light can be increased in an effort to provide sufficient light to the bottom or central portion of the fermenter. However, diffused light has a large attenuation factor when traveling in the culture solution. To increase the amount of emitted light sufficiently to ensure that enough light reaches the bottom of the fermenter, energy consumption (for example, power) increases markedly.

After the amount of light shining on a photosynthetic microorganism reaches a given level, the reaction rate of photosynthesis does not increase even if the amount of irradiating light increases. So, increasing the amount of light emitted by a light source so that more light reaches the bottom of the fermenter does not improve photosynthesis near the liquid level of the culture solution, and the overall energy efficiency for the microorganism culture (e.g., the amount of photosynthetic microorganism that can be cultured by energy consumption per unit (for example, dry weight)) decreases. And when the amount of light irradiating a photosynthetic microorganism increases excessively, photosynthetic activity may decreases due to photoinhibition, inhibiting growth of the photosynthetic microorganism near the liquid level of the culture solution. As described above, increasing the amount of light emitted by the light source does little to increase either the space efficiency or the energy efficiency for the photosynthetic microorganism culture.

### SUMMARY OF THE INVENTION

The present invention has been made in view of the above circumstances. An object of the present invention is to provide a photosynthetic microorganism culture apparatus that can hygienically culture a photosynthetic microorganism with good space efficiency and energy efficiency, using artificial light.

(1) To achieve the above object, according to one aspect of the present invention, a photosynthetic microorganism culture apparatus includes: a fermenter in which a culture solution including a photosynthetic microorganism is stored, a light incidence region through which light enters the culture solution that is being formed in the fermenter; one or a plurality of parallel light sources that emit parallel light; a reflection mechanism that reflects the parallel light emitted from the parallel light source in a predetermined direction as a single source of reflected parallel light (collimated light); and a controller that causes the reflected parallel light to travel periodically in a previously-fixed direction to irradiate the light incidence region with the reflected parallel light.

In one aspect of the present invention, the parallel light emitted from the one or plurality of parallel light sources is reflected in the predetermined direction as the a single source of reflected parallel light. The reflected parallel light travels periodically in the previously-fixed direction, and the light incidence region of the fermenter is irradiated with the reflected parallel light. The whole light incidence region is irradiated with the reflected parallel light. Compared with the case in which the culture solution is irradiated with diffused light emitted from a diffused light source, the attenuation factor of a photon flux density is markedly suppressed when the light travels in the culture solution. Even if the amount of photosynthetic microorganism per unit volume of the culture solution (hereinafter referred to as the microorganism density) increases, light having sufficient photon flux density for photosynthesis is delivered to the region distant from the light incidence region. Accordingly, photosynthetic microorganisms that are relatively far from the light incidence region (e.g., deep within the fermenter) still receive enough light to effectively photosynthesize. As a result, the space efficiency for the microorganism culture is improved. The energy efficiency for the microorganism culture is improved by suppressing the attenuation of the photon flux density. The present invention can work not only where a fermenter is irradiated with the reflected parallel light but also the case that a plurality of fermenters are irradiated with the reflected parallel light.

(2) In the photosynthetic microorganism culture apparatus, it may be preferred that the parallel light source includes a laser diode.

In that configuration, because the parallel light source includes the laser diode, the light incidence region is irradiated with parallel light having high straightness. Using a so-called semiconductor laser device as the parallel light source provides high energy conversion efficiency. As a result, the energy efficiency for the culture of the photosynthetic microorganism is improved.

(3) In the photosynthetic microorganism culture apparatus, the controller may control the parallel light source such that the culture solution is intermittently irradiated with the reflected parallel light at a frequency corresponding to the time necessary for the photosynthetic light reaction in the photosynthetic microorganism.

In that configuration, the photosynthetic microorganism is not irradiated with the light in a period in which the light is not required for photosynthesis, and the photosynthetic microorganism is irradiated with the light only in a period in which the light is required for photosynthesis. As a result, a photosynthesis rate per unit light amount increases to improve the energy efficiency for the culture of the photosynthetic microorganism.

(4) In the photosynthetic microorganism culture apparatus, the controller may control the reflection mechanism such that the reflected parallel light is reflected in a period corresponding to the time necessary for a photosynthetic light reaction in the photosynthetic microorganism.

In this case, the photosynthetic microorganism is not irradiated with the light in the period in which the light is not required for photosynthesis, and the photosynthetic microorganism is irradiated with the light only in the period in which the light is required for photosynthesis. As a result, a photosynthesis rate per unit light amount increases to improve the energy efficiency for the culture of the photosynthetic microorganism.

(5) In the photosynthetic microorganism culture apparatus, it may be preferred that the controller control the reflection mechanism to form a reflection route. The reflected parallel light moves to an inside of the light incidence region again after moving from the inside to an outside of the light incidence region in the reflection route. Preferably the controller controls the parallel light source to stop emission of the parallel light when the reflected parallel light moves from the inside to the outside of the light incidence region, and to start emission of the parallel light again when an optical axis of the reflected parallel light moves from the outside to the inside of the light incidence region assuming that irradiation of the reflected parallel light is continued.

In the configuration, the reflection route of the reflected light is inverted after the reflected light passes through the end portion of the light incidence region from the inside to the outside. The irradiation amount of the reflected light relatively increases near an inversion point of the reflection route. When the inversion point of the reflection route is located in the end portion of the light incidence region, the irradiation amount of the reflected light increases near the end portion. Because the inversion point of the reflection route is located outside the light incidence region, the irradiation amount of the reflected light is prevented from increasing near the end portion of the light incidence region. Accordingly, the irradiation amount of the reflected light is equalized in the whole light incidence region. The parallel light source does not emit the parallel light while the reflected parallel light travels on the outside of the light incidence region. As a result, the parallel light source is prevented from emitting unnecessary parallel light, and the energy efficiency for the culture of the photosynthetic microorganism is improved.

(6) In the photosynthetic microorganism culture apparatus, it may be preferred that the reflection mechanism include: a mirror in which a reflection angle is variable, and the mirror can reflect the light as a single source of reflected parallel light; and a driver that changes the reflection angle of the mirror.

In that configuration, the light source does not turn, but the mirror (which can be light-weight) turns to irradiate the whole light incidence region. The whole light incidence region is accurately irradiated with the reflected parallel light in a desired short period.

(7) In the photosynthetic microorganism culture apparatus, the light incidence region of the fermenter may be at the top, and a bottom contour may be set based on a traveling direction of the reflected parallel light incident on the light incidence region along a contour of the light incidence region.

In that configuration, the whole culture solution stored in the fermenter is irradiated with the reflected parallel light. The culture solution is used to culture the photosynthetic microorganism without waste. For example, the fermenter may be formed into a truncated cone shape in a case where the liquid level of the fermenter is irradiated with the light from above. Compared with a fermenter formed in a rectangular parallelepiped shape that has the same depth and the same bottom width (diameter), the volume of a fermenter having the truncated cone shape is significantly smaller than the volume of the fermenter having the rectangular parallelepiped shape. Accordingly, the amount of culture solution used to culture the photosynthetic microorganism is decreased.

(8) According to another aspect of the present invention, a lighting device can be provided in a fermenter in which a photosynthetic microorganism culture solution is stored and the light incidence region through which light enters the culture solution is formed in the fermenter. The lighting device includes: one or a plurality of parallel light sources that emit parallel light; a reflection mechanism that reflects the parallel light emitted from the parallel light source in a predetermined direction as reflected parallel light from a single source; and a controller that causes the reflected parallel light reflected by the reflection mechanism to travel periodically in a previously-fixed direction or path to irradiate the light incidence region with the reflected parallel light.

In this embodiment of the invention, the parallel light emitted from the one or plurality of parallel light sources is reflected by the reflection mechanism in the predetermined direction as parallel light from a single source. The parallel light travels periodically in the previously-fixed direction, and the whole light incidence region of the fermenter is irradiated with the reflected parallel light. Compared with an arrangement in which the culture solution is irradiated with diffused light emitted from a diffused light source, the attenuation factor of a photon flux density is markedly suppressed when the light travels in the culture solution. For example, the space efficiency is improved in the case that a vertically long fermenter is used. The energy efficiency for the photosynthetic microorganism culture is improved by suppressing the attenuation of the photon flux density.

(9) According to still another aspect of the present invention, a method for culturing a photosynthetic microorganism using a fermenter in which a photosynthetic microorganism culture is stored and the light incidence region through which light enters the culture solution is formed in the fermenter includes the steps of: emitting parallel light from one or a plurality of parallel light sources; reflecting the parallel light emitted from the parallel light source in a predetermined direction as a single source of parallel light using a reflection mechanism that can variably control a reflection direction; and controlling the reflection mechanism such that the reflected parallel light reflected by the reflection mechanism is caused to travel periodically in a previously-fixed direction.

In this aspect of the present invention, the parallel light emitted from the one or plurality of parallel light sources is reflected in the predetermined direction as the reflected parallel light by the reflection mechanism. The reflected parallel light travels periodically in the previously-fixed direction, and the whole light incidence region of the fermenter is irradiated with the reflected parallel light. Compared with a situation in which the culture solution is irradiated with diffused light emitted from a diffused light source, the attenuation factor of the photon flux density is markedly suppressed when the light travels in the culture solution. For example, the space efficiency is improved in the case that a vertically long fermenter is used. The energy efficiency for the photosynthetic microorganism culture is improved by suppressing the attenuation of the photon flux density.

(10) As described above, the present invention provides a photosynthetic microorganism culture apparatus that can hygienically culture the photosynthetic microorganism with the good space efficiency and energy efficiency using the artificial light.

### BRIEF DESCRIPTION OF THE DRAWINGS

FIG. 1 is a perspective view schematically illustrating an entire configuration of a photosynthetic microorganism culture apparatus 10 according to an embodiment of the present invention;

FIG. 2 is a front view illustrating a lighting device 12 including a light source device 31 and a reflection mechanism 32;

FIG. 3 is a perspective view schematically illustrating a structure of the lighting device 12;

FIG. 4 is a functional block diagram illustrating a schematic configuration of a control system of the lighting device 12;

FIG. 5 is a functional block diagram illustrating a schematic configuration of a control device 13;

FIG. 6 is a flowchart of lighting processing performed by the control device 13;

FIG. 7 is a plan view schematically illustrating an example of a reflection route 23 in a light incidence region 22;

FIG. 8 is a plan view schematically illustrating another example of the reflection route 23 in the light incidence region 22;

FIG. 9 is an enlarged plan view schematically illustrating a part of the reflection route 23 in the light incidence region 22;

FIG. 10 is a graph illustrating a measurement result of a photon flux density of parallel light 21 when the light incidence region 22 is irradiated along a line VI-VI in FIG. 7;

FIG. 11 is a graph illustrating a measurement result of a photon flux density of parallel light 21 when the light incidence region 22 is irradiated along the line VI-VI in FIG. 7 and the control device 13 causes the light source device 31 to stop emitting the laser beam 33 when the irradiation spot 24 reaches the endpoint 23B and to resume emitting the laser beam when the irradiation spot reaches the endpoint 23C; and

FIG. 12 is a perspective view schematically illustrating an entire configuration of a photosynthetic microorganism culture apparatus 10A according to a modification of the embodiment.

### DESCRIPTION OF THE PREFERRED EMBODIMENTS

A preferred embodiment of the present invention will be described with reference to the drawings. The embodiment of the present invention is described below only by way of example, and various changes can be made without departing from the scope of the present invention.

### Overall Configuration of a Photosynthetic Microorganism Culture Apparatus 10

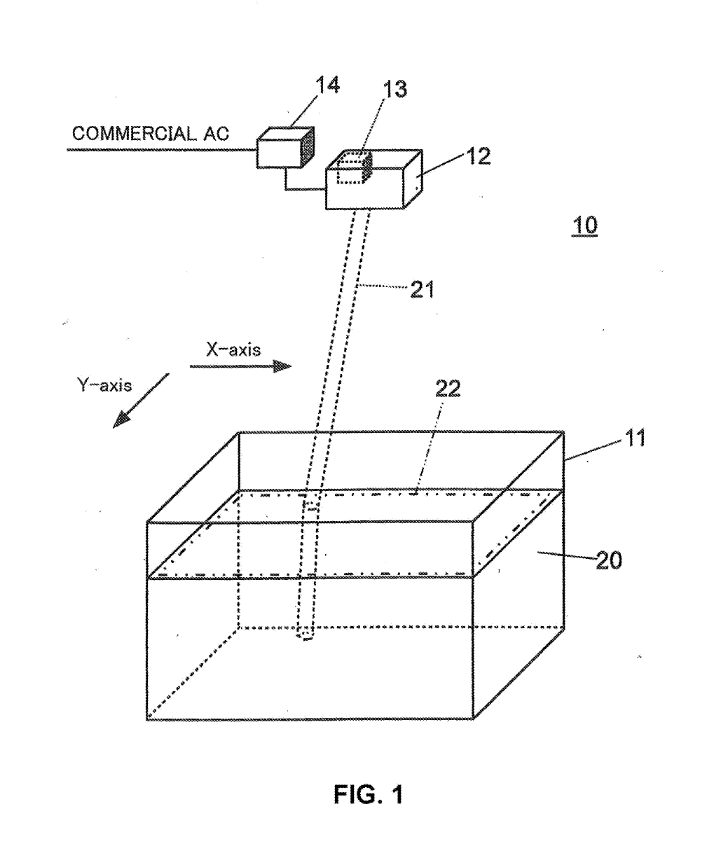

FIG. 1 illustrates a photosynthetic microorganism culture apparatus 10 that includes a fermenter 11 in which a culture solution 20 is stored. A lighting device 12 irradiates the culture solution 20 with parallel light 21 (collimated light). A control device 13 controls the lighting device 12, and a power supply device 14 powers the lighting device 12. The control device 13 may be integral with the lighting device 12, or separated from the lighting device 12. Similarly, the power supply device 14 may be integral with the lighting device 12, or separated from the lighting device 12. The culture solution 20 includes a liquid medium and a photosynthetic microorganism (not illustrated). The photosynthetic microorganism culture apparatus 10 may include a gas supply unit (not illustrated), such as a gas pump and a blower. The gas supply unit supplies gas such as carbon dioxide and oxygen to the culture solution 20 stored in the fermenter 11. The apparatus may also include a temperature maintaining unit (not illustrated), such as a heater and a cooling fan, which maintains the culture solution 20 at a temperature suitable for growing the photosynthetic microorganism. The photosynthetic microorganism culture apparatus 10 may also include a stirring device (not illustrated) that stirs the culture solution 20.

### The Fermenter 11

There is no particular limitation to a shape of the fermenter 11. FIG. 1 illustrates a fermenter 11 that generally has the shape of a rectangular parallelepiped container with an open top. By way of example, the lighting device 12 is disposed above a liquid level of the culture solution 20 stored in the fermenter 11. In the example illustrated in FIG. 1, a light incidence region 22 is considered to be at the liquid level of the culture solution 20. The parallel light 21 with which the culture solution 20 is irradiated goes into the fermenter 11 through the light incidence region 22.

There is no particular limitation to the construction material used to make the fermenter 11. The illustrated fermenter 11 is made of a transparent material such as an acrylic resin and glass or an opaque material such as an FRP (Fiber Reinforced Plastics) and stainless steel. If the fermenter 11 is made of transparent material, the lighting device 12 can be disposed on a side of the fermenter 11. In this case, the light incidence region 22 can be considered to be a sidewall of the fermenter 11. The parallel light 21 is refracted at the liquid level of the culture solution 20 when going into the culture solution 20 through the light incidence region 22.

The culture solution 20 is stored in the fermenter 11. The culture solution 20 includes the liquid medium and the photosynthetic microorganism. To prepare the culture, the liquid medium can be subjected to heat-sterilizing processing and cooled, and then a liquid including the photosynthetic microorganism (such as a microalga) can be mixed in the liquid medium. The liquid medium may properly include nitrogen, potassium, phosphorus, calcium, magnesium, manganese, and iron to promote the growth of the photosynthetic microorganism. Chlorella (the genus Chlorella), spirulina (the genus Arthrospira), the genus Euglena (euglena), the genus Chlamydomonas, the genus Scenedesmus, and the genus Ankistrodesmus can be cited as examples of photosynthetic microorganisms that can be included in the culture solution 20.

### The Light Incidence Region 22

When the light incidence region 22 is at the liquid level of the culture solution 20, a contour of the light incidence region 22 may be matched with a contour at the liquid level of the culture solution 20, or set inside that contour. When the fermenter 11 has a transparent sidewall, it may be desirable to set the contour of the light incidence region 22 at the inside of the contour at the liquid level of the culture solution 20 so that the parallel light 21 is not emitted to the outside of the fermenter 11 through the sidewall of the fermenter 11.

It may be desirable that the light incidence region 22 have a planar shape. The light incidence region 22 may have a curved shape if the sidewall of the fermenter 11 is formed only by a curved surface and the light incidence region 22 is on the sidewall. In the case that the light incidence region 22 is on the liquid level of the culture solution 20, a mirror may be formed in an inner surface of the sidewall of the fermenter 11 and in an inner surface of the bottom of the fermenter 11. In that way, parallel light 21 going into the culture solution 20 through the light incidence region 22 can be reflected toward the inside of the fermenter 11 by the inner surface of the sidewall and the inner surface of the bottom of the fermenter 11.

### The Lighting Device 12

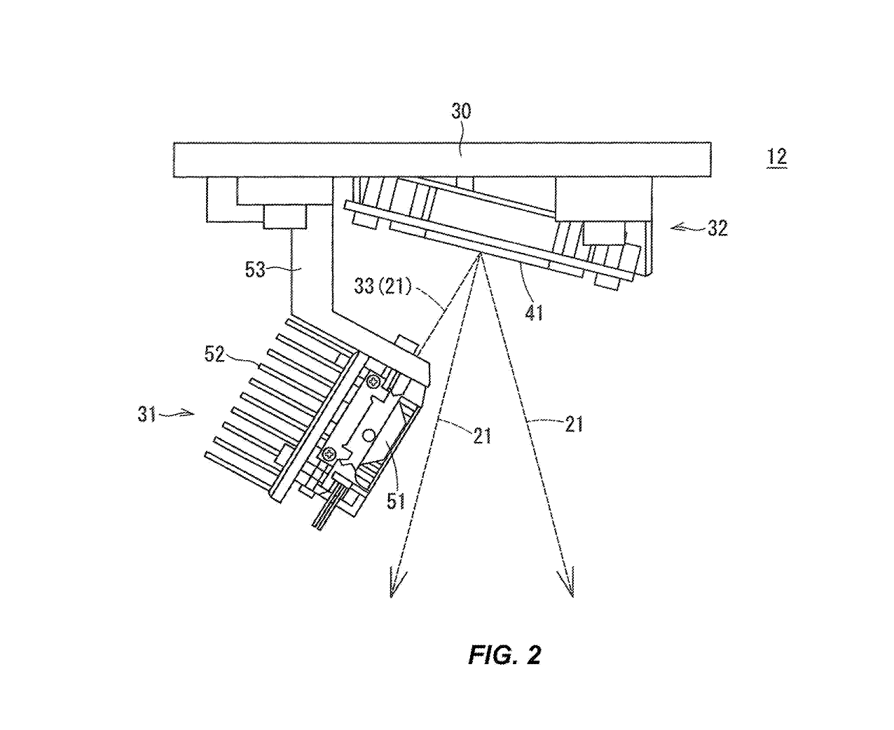

The lighting device 12 illustrated in FIG. 2 includes a light source device 31, a reflection mechanism 32, and a base plate 30 on which the light source device 31 and the reflection mechanism 32 are mounted. The light source device 31 of the invention includes a one or a plurality of laser diodes 51. The light source device 31 may include only one laser diode 51, and typically the light source device 31 will include one to ten laser diodes 51. Each laser diode 51 is a parallel light source. The light source device 31 may include a heat sink 52 that radiates heat of the laser diode 51. The light source device 31 is fixed to the base plate 30 by a support tool 53.

### The Light Source Device 31

As noted above, the light source device 31 includes a laser diode 51. The laser diode 51 emits a laser beam 33 having a wavelength suitable for the photosynthesis by the photosynthetic microorganism in the culture solution 20. For example, when the photosynthetic microorganism is chlorella, the laser diode 51 used in the light source device 31 may emit a laser beam 33 that has wavelength λ1 (for example, desirably 440 nm≦λ1≦460 nm, more desirably 430 nm≦λ1≦480 nm) in which a center wavelength is 450 nm. The laser diode 51 used in the light source device 31 emits the laser beam 33 having a wavelength λ2 (for example, desirably 650 nm≦λ2≦670 nm, more desirably 645 nm≦λ2≦685 nm) in which the center wavelength is 660 nm. The laser diode 51 used in the light source device 31 emits the laser beam 33 having the center wavelength of 560 nm to 620 nm.

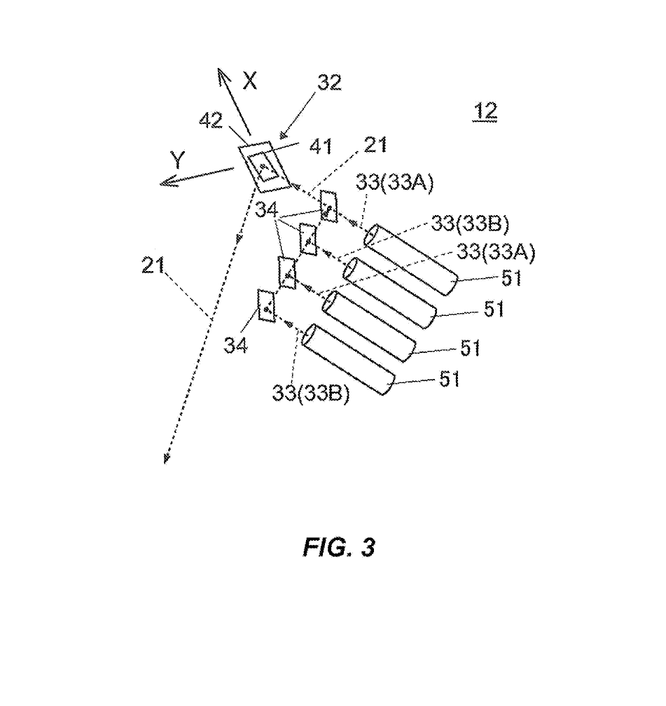

The lighting device 12 illustrated in FIG. 3 includes the two laser diodes 51 that each emit a laser beam 33A having wavelength λ1 and the two other laser diodes 51 that each emit a laser beam 33B having wavelength λ2. The laser beams 33 emitted from the plurality of laser diodes 51 are reflected by semitransparent collective mirrors 34 to create parallel light 21 collimated light). Different laser diodes 51 may be used to provide different wavelengths. For example, the lighting device 12 may include one laser diode 51 that emits laser beam 33A having wavelength λ1 and another laser diode 51 that emits laser beam 33B having wavelength λ2.

The lighting device 12 may include laser diodes 51 that emit not different wavelengths but the one wavelength (for example, wavelength λ2). In this case, one or a plurality of laser diodes 51 are provided in the lighting device 12. The number of laser diodes 51 that emit the laser beam 33 having one wavelength may be larger than the number of laser diodes 51 that emit the laser beam 33 having another wavelength. For example, the lighting device 12 may have two laser diodes 51 that emit the laser beam 33A, and three laser diodes 51 that emit the laser beam 33B.

### The Reflection Mechanism 32

The reflection mechanism 32 illustrated in FIG. 3 includes a mirror 41 that reflects parallel light 21 and a driver 42 that drives the mirror 41. For example, the reflection mechanism 32 can be a MEMS (Micro Electro Mechanical System). Because the structure of a MEMS is well known, the description is omitted.

### The Mirror 41

By way of example, the mirror 41 can be constructed with a silicon micro mirror or a metal mirror. The mirror 41 is supported by a support mechanism (not illustrated). The support mechanism turns the mirror 41 about two axes (an X-axis and a Y-axis) that are orthogonal to each other. The mirror 41 irradiates the whole light incidence region 22 with reflected parallel light from a parallel light beam 21 provided by laser diodes 51.

### The Driver 42

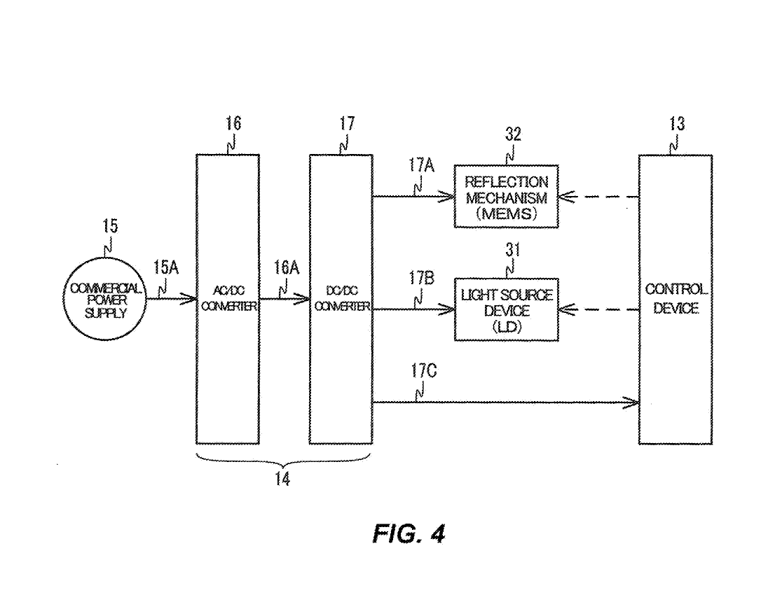

The driver 42 includes an actuator and a driver circuit (not illustrated) that turn the mirror 41 about the two axes (the X-axis and the Y-axis). The control device 13 controls the power supply device 14 based on a control command. In turn, the power supply device 14 supplies a DC voltage 17A to the driver circuit. The DC voltage 17A in the driver circuit drives the actuator. In the illustrated embodiment, the driver 42 includes a moving-coil-type actuator. The moving-coil-type actuator includes two coils that are provided in the support mechanism that supports the mirror 41, two elastic members that bias the support mechanism to restore the mirror 41 to a default state, and two pairs of fixedly-disposed permanent magnets. When the coil is energized, Lorentz force is generated by interaction with a magnetic field of the permanent magnet, and that Lorentz force is applied to the support mechanism, against the force of the biasing elastic member. The mirror 41 tilts to the angle at which the Lorentz force and biasing force of the elastic member balance each other. Using the DC voltage 17A (see FIG. 4), the driver circuit generates a pulse current in which an on state and an off state are alternately switched at a frequency in response to the control command from the control device 13. The driver circuit generates the pulse current having either one kind of frequency or two kinds of frequencies. A pulse current having one kind of the frequency is supplied to one of the two coils, and the pulse current having two kinds of the frequencies is supplied to both coils. As a result, the mirror 41 receives a restoring force from the elastic member in at least one of the turning directions about the X-axis and the Y-axis, and vibrates with a period corresponding to the one kind of the frequency or the two kinds of the frequencies. The driver circuit can also be provided in the control device 13.

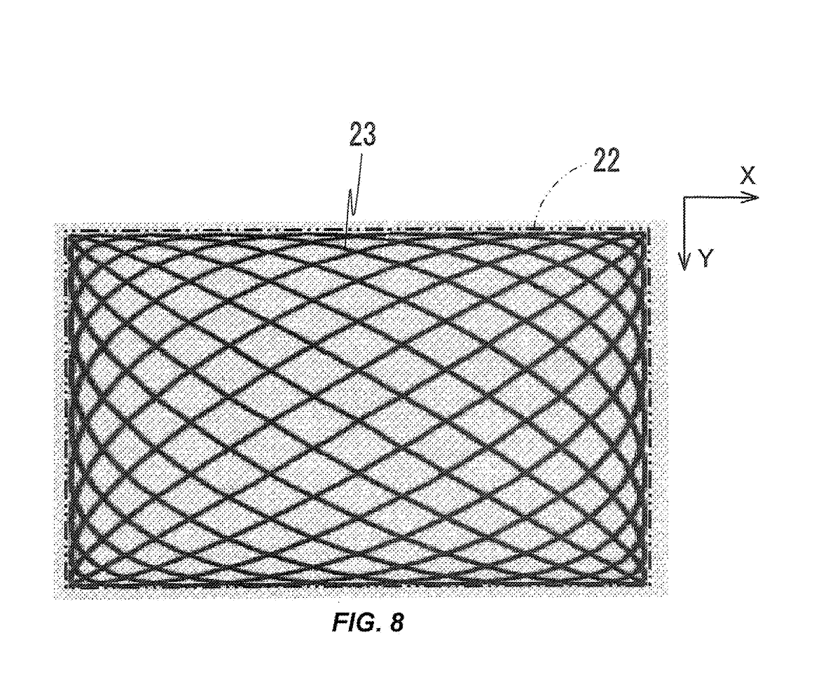

In this embodiment, a resonance frequency in the turning direction of the mirror 41 about the X-axis differs from a resonance frequency in the turning direction of the mirror 41 about the Y-axis. The pulse current having the frequency corresponding to the resonance frequency in each turning direction is supplied to each of the two coils. Therefore, the reflected light from the parallel light beam 21 travels so as to draw a Lissajous figure in the light incidence region 22 (see FIG. 8). The mirror 41 may be driven by a pulse current having a frequency except for the resonance frequency. Compared with the case in which the mirror 41 is driven by a pulse current whose frequency corresponds with the resonance frequency, energizing the coil to vibrate the mirror to the identical amplitude using a pulse current whose frequency has the resonance frequency subtracted out requires only a fraction of the energy (several times to several tens of times more energy required using a pulse current whose frequency corresponds with the resonance frequency). It is necessary to properly set the resonance frequency of the reflection mechanism 32 to reduce running cost of the photosynthetic microorganism culture apparatus 10. Therefore, the desirable period T (to be described later) is obtained.

A moving-magnet-type actuator, an electrostatic-type actuator, and a piezoelectric-type actuator may be used in the driver 42. A moving-magnet-type actuator may include a magnet in the mirror 41 and a fixedly-disposed coil, and turns the mirror 41 by energizing the coil. An electrostatic-type actuator may include a fixedly-disposed electrode and an electrode in the mirror 41 that has an opposite polarity, and changes an angle of the mirror 41 by the electrostatic force that is generated by applying a voltage between the electrodes. By way of example, a piezoelectric-type actuator may include a lead zirconate titanate (PZT) thin film that supports the mirror 41. In such a piezoelectric-type actuator, voltage is applied to the thin film to deform the thin film, thereby changing the angle of the mirror 41.

### The Control System of Lighting Device 12

The control device 13 illustrated in FIG. 4 controls the light source device 31 and the reflection mechanism 32 of the lighting device 12. The power supply device 14 includes an AC-DC converter 16 and a DC-DC converter 17. The AC-DC converter 16 converts an AC voltage 15A supplied from a commercial power supply 15 into a DC voltage 16A. The DC-DC converter 17 transforms the DC voltage 16A, and outputs the transformed DC voltage 16A as DC voltages 17A, 17B, and 17C. The DC voltage 17A is supplied to the reflection mechanism 32, the DC voltage 17B is supplied to the light source device 31, and the DC voltage 17C is supplied to the controller 13. The DC voltage 17A may be equal to or different from the DC voltages 17B and 17C.

### The Control Device 13

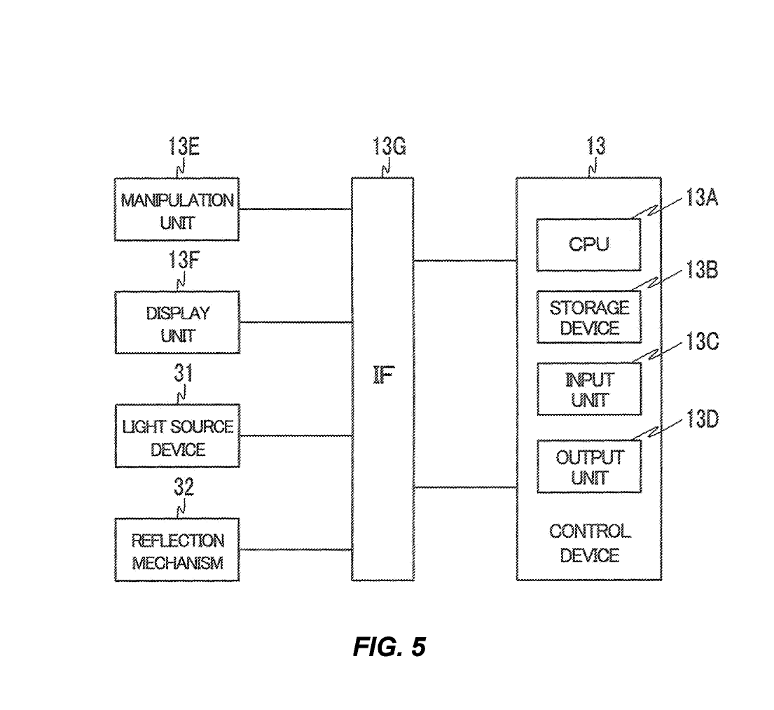

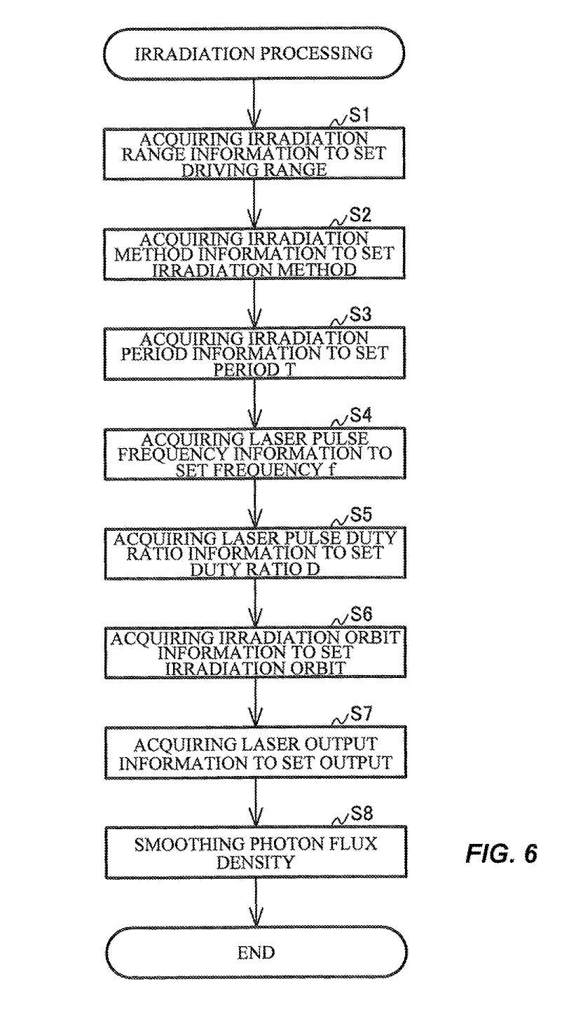

As illustrated in FIG. 5, the control device 13 includes a CPU (Central Processing Unit) 13A, a storage device 13B, an input unit 13C, and an output unit 13D. The control device 13 can be constructed with an electronic circuit board. The storage device 13B includes a ROM (Read Only Memory) and a RAM (Random Access Memory), and stores a control program that causes the CPU 13A to perform irradiation processing (see FIG. 6). The input unit 13C is connected to a manipulation unit 13E through an interface 13G. A user inputs various pieces of information through the manipulation unit 13E. The output unit 13D outputs a control command—a processing result of the irradiation processing—to the light source device 31 and to the reflection mechanism 32 through the interface 13G. The output unit 13D is connected to a display unit 13F through the interface 13G. The display unit 13F displays the information input from the input unit 13C.

As illustrated in FIG. 6, during irradiation processing, the CPU 13A of the control device 13 acquires irradiation range information that is input to the manipulation unit 13E by the user, and sets a driving range of the mirror 41 based on the irradiation range information (S1). The irradiation range information includes information on a size of the light incidence region 22 in X-axis and Y-axis directions, information on a vertical distance between the lighting device 12 and the light incidence region 22, and information on a position of the lighting device 12 in the X-axis and Y-axis directions. For example, the CPU 13A sets a rotation angle range of the mirror 41 about the X-axis and the Y-axis based on pieces of this information. The rotation angle range is adjusted by a value (maximum current value) of the current (alternating current) passed through the coil.

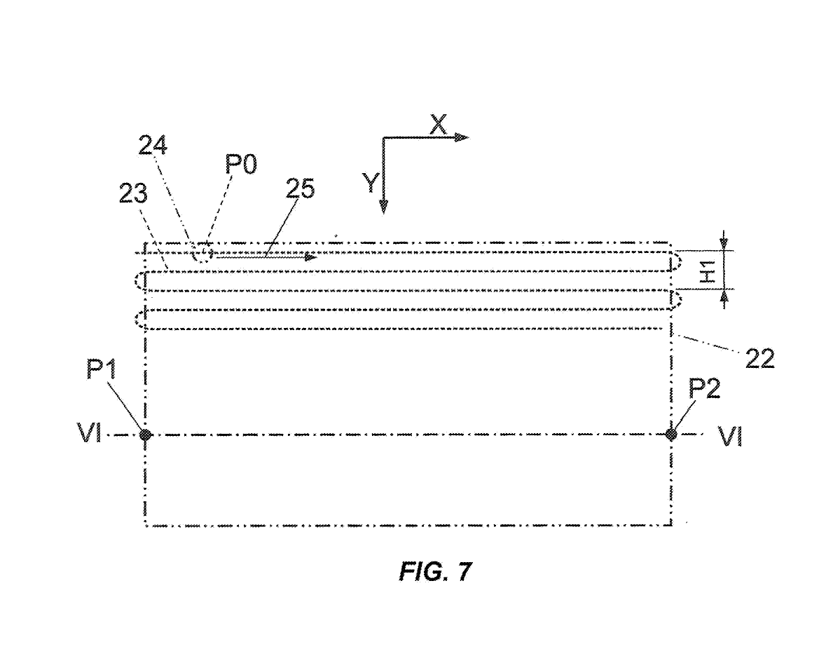

During irradiation processing, the CPU 13A also acquires irradiation method information that is input to the manipulation unit 13E by the user, and performs processing using an irradiation method (S2). Examples of irradiation methods include a scanning line method and a Lissajous method. In the scanning line method, which is comparable to the method of display of an image on a television screen, the light incidence region 22 is divided into a plurality of rectangular regions in the Y-axis direction, and the rectangular regions are sequentially irradiated with the parallel light 21 in the X-axis direction (see FIG. 7). In the Lissajous method, the light incidence region 22 is irradiated with the parallel light 21 such that the Lissajous figure (see FIG. 8) is drawn.

During irradiation processing, the CPU 13A processes acquired irradiation period information to set a period T (S3), processes acquired laser pulse frequency information to set a frequency f at which the laser diode 51 of the light source device 31 is blinked (S4), and processes acquired laser pulse duty ratio information to set a duty ratio D (S5). The user inputs the irradiation period information (period T), the laser pulse frequency information (frequency f), and the laser pulse duty ratio information (duty ratio D) to the manipulation unit 13E.

### The Period T

As an example, when the scanning line method is used as the irradiation method, and the light incidence region 22 has a rectangular shape, the reflected light of the parallel light beam 21 travels along a reflection route 23 as illustrated in FIG. 7. The CPU 13A transmits the control command to the reflection mechanism 32 in order to control the reflected light. An irradiation spot 24 in which the light incidence region 22 is irradiated with the reflected light travels in a direction indicated by the arrow 25 along the reflection route 23. According to the reflection route 23 in FIG. 7, the irradiation spot 24 reciprocates in the X-axis direction along a long side of the light incidence region 22. The movement of the irradiation spot 24 in the X-axis direction corresponds to the turning of the mirror 41 about the Y-axis (see FIG. 3).

As illustrated in FIG. 7, the irradiation spot 24 moves in the Y-axis direction by a width H1 while making a round trip in the X-axis direction. The width H1 is equal to a diameter of the irradiation spot 24. The movement of the irradiation spot 24 in the Y-axis direction corresponds to the turning of the mirror 41 about the X-axis (see FIG. 3). The period T is period of time that passes from the time that the irradiation spot 24 passes through any point P0 to the time that the irradiation spot next passes again through that point P0.

### The Frequency f

The frequency f is the frequency at which the laser diode 51 of the light source device 31 blinks. It is known that a photosynthesis rate in butterhead lettuce increases when exposed to intermittent light having the frequency f of 2500 Hz (see Report of Resource Survey Subcommittee of Science Council, “2-5 Problems with photosynthesis reaction and pulse irradiation LED plant factory” in chapter 2 “Light contributing to wealthy life” of “Promotion of creative science and technology using light source, toward sustainable “Century of Light””, the Ministry of Education, Culture, Sports, Science, and Technology, Sep. 5, 2007).

Photosynthetic microorganisms such as chlorella include a photochemical system like butterhead lettuce and chlorophyll. Accordingly, when culturing a photosynthetic microorganism such as chlorella, it may be desirable to set the frequency f in the range of 2500 Hz. For example, it may be desirable to set the frequency f in the range of 2000 Hz to 3000 Hz. Preferably, the period T is set to 400 μsec (=( 1/2500) sec). For example, it may be desirable to set the period T in the range of 300 μsec to 500 μsec. At this point, the laser diode 51 may blink or need not blink at the frequency f.

### The Duty Ratio D

In a cycle in which the culture solution 20 is first irradiated with light and then is left dark in advance of the next light period, the duty ratio D is the ratio of a light period to the total (light and dark) period. It is known that a growth rate of butterhead lettuce increases significantly when the frequency f is set to 2500 Hz and the duty ratio D is 33% (see Report of Resource Survey Subcommittee of Science Council, “2-5 Problems with photosynthesis reaction and pulse irradiation LED plant factory”).

To increase the growth rate of photosynthetic microorganisms such as chlorella, it may be desirable to use a duty ratio D of about 33% when the frequency f is set in the desirable range. For example, a duty ratio D in the range of 25% to 41% may be desirable. Such a duty ratio may suppress energy consumption and improve the energy efficiency.

During irradiation processing, the CPU 13A processes acquired irradiation orbit information to set an irradiation orbit (S6), processes acquired laser output information to set an output of the laser diode 51 of the light source device 31 (S7), and performs processing of smoothing photon flux density (S8). The user inputs the irradiation orbit information and the laser output information to the manipulation unit 13E.

### The Irradiation Orbit Information

When the photosynthetic microorganisms are irradiated using the scanning line method, the irradiation orbit information may include information (such as a diameter of the irradiation spot 24) on the width H1. When the Lissajous method is used, the irradiation orbit information may include information on the turning frequency of the mirror 41 about the X-axis and information on the turning frequency of the mirror 41 about the Y-axis.

### The Laser Output Information

The output of the laser diode 51 of the light source device 31 is preferably adjusted based on the kind of photosynthetic microorganism being used, the vertical distance between the lighting device 12 and the light incidence region 22, the sizes of the light incidence region 22 in the X-axis and Y-axis directions, and the depth of the fermenter 11.

### The Photon Flux Density Smoothing Processing

To smooth the photon flux density of the parallel light 21 that is used to irradiate the light incidence region 22, the speed at which the irradiation spot 24 travels along the reflection route 23 is kept generally constant. This can be done using the procedure described next.

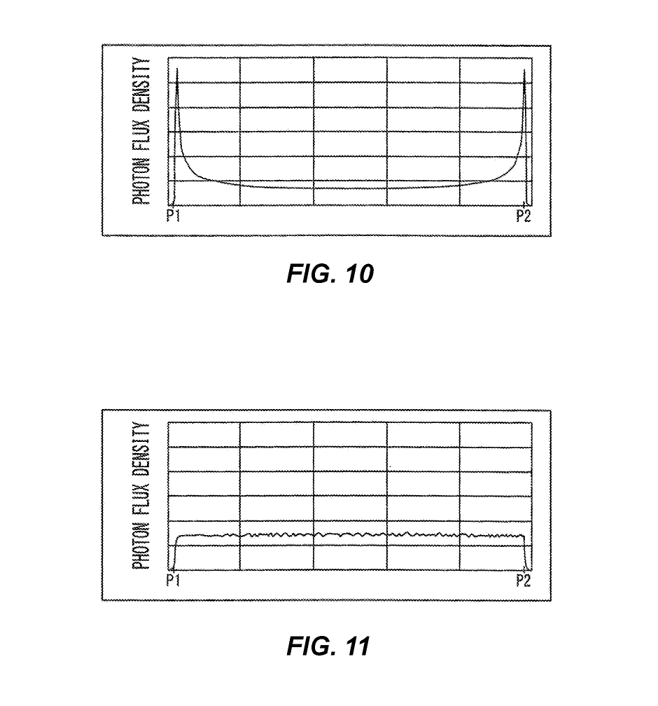

The driver circuit supplies a pulse current to at least one of the coils, whereby the mirror 41 vibrates in at least one of the turning directions about the X-axis and the Y-axis. When the mirror 41 vibrates, the rotational speed of the mirror 41 increases in the temporal midpoint of the vibration, and thus the speed at which the reflected light moves across the light incidence region also increases. When the mirror 41 vibrates in the turning direction about the Y-axis, the speed of the irradiation spot 24 increases in a central portion of the light incidence region 22 in the X-axis direction. As a result, the photon flux density decreases in that portion of the light incidence region 22. (see FIG. 10). At this juncture, it may be preferred to use the driver circuit to loosen the increase in the pulse current input to the coil as a means to increase the photon flux density in the central portion of the light incidence region 22 in the X-axis direction. By restraining the speed at which the irradiation spot traverses the central portion of the light incidence 22 in the X-direction, the decrease of the photon flux density in that region can be reduced or prevented. The same holds true for the Lissajous method (see FIG. 8).

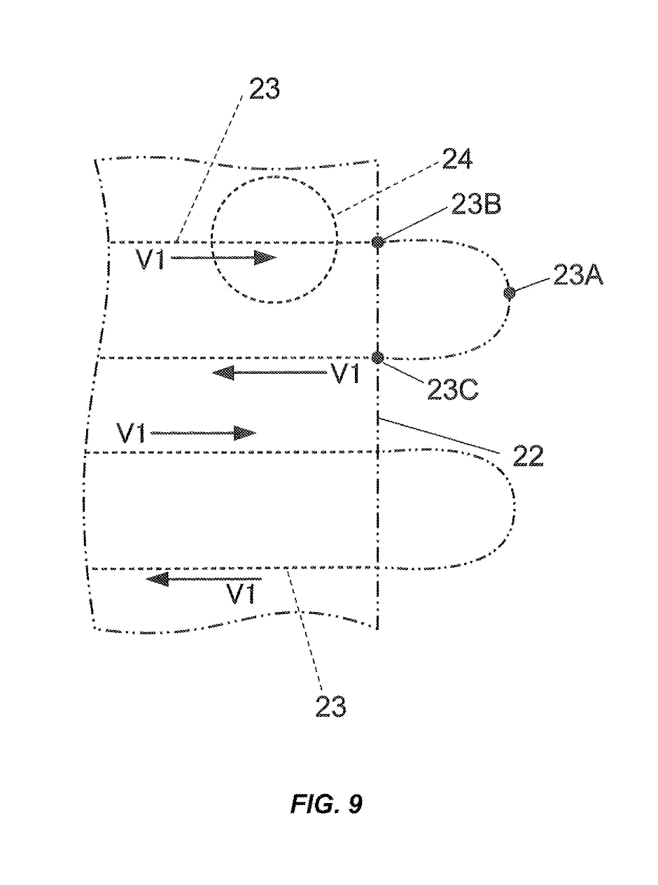

As illustrated in FIG. 9, to smooth the photon flux density of the parallel light 21 that irradiates the light incidence region 22, it may be preferred to position an inversion point 23A of the reflection route 23 not in an end portion of the light incidence region 22 but outside the light incidence region 22. FIG. 9 illustrates an example in which the irradiation spot 24 reciprocates in the X-axis direction by the scanning line method. When the Lissajous method is used, the irradiation spot 24 reciprocates in both the X-axis and Y-axis directions. In that case, it may be preferred to position the inversion points in both the X-axis and Y-axis directions outside the light incidence region 22. When the inversion point 23A of the reflection route 23 is disposed in the end portion of the light incidence region 22, as illustrated in FIG. 10, the photon flux density increases markedly at endpoints P1 and P2 (see FIG. 7) of the light incidence region 22. When the inversion point 23A of the reflection path 23 is disposed outside the light incidence region 22, the speed at which the irradiation spot 24 moves across the light incidence region does not decrease until the irradiation spot 24 reaches the end portion of the light incidence region 22. Because the speed at which the irradiation spot 24 moves is kept constant, an irradiation amount (photon flux density) of the light incidence region 22 with the parallel light 21 is smoothed across the whole light incidence region 22.

When the irradiation spot 24 moves from inside the light incidence region 22 to outside that region (through the endpoint 23B (see FIG. 9)), it may be preferred to use the control device 13 to stop the laser beam 33 emitted by the laser diode 51 at the endpoint 23B. (It is assumed that the laser diode 51 emits the laser beam 33.) When an optical axis of the laser beam 33 moves from outside the light incidence region back into the light incidence region (through the endpoint 23C), it may be preferred to use the control device 13 to resume the laser beam 33 emitted by the laser diode 51. The process of turning on or off the laser diode 51 when the irradiating light reaches the endpoints 23B and 23C is performed based on a calculation result of the CPU 13A. The CPU 13A calculates the time when the irradiation spot 24 (or the optical axis of the irradiation spot 24) passes through the endpoints 23B and 23C based on the vibration frequency and amplitude of the mirror 41 in the X-axis and Y-axis directions. When the reflection route 23 is fixed, the time when the irradiation spot 24 (or the optical axis of the irradiation spot 24) passes through the endpoints 23B and 23C on the reflection route 23 is easily calculated based on the vibration frequency and amplitude of the mirror 41. Through the above pieces of processing, the emission of the unnecessary light by the laser diode 51 is prevented, improving the energy efficiency.

### The Operation of Photosynthetic Microorganism Culture Apparatus 10

As illustrated in FIG. 1, while the culture solution 20 is stored in the fermenter 11, the power supply device 14 supplies power to the lighting device 12 and the control device 13 to begin operation of start the photosynthetic microorganism culture apparatus 10. When operation has begun, the laser diode 51 of the light source device 31 start emitting the laser beam 33. If the light source device 31 includes a plurality of laser diodes 51, then the laser beams 33 emitted from the laser diodes 51 are collected by the collective mirrors 34 to form parallel light 21. The parallel light 21 is incident on the reflection mechanism 32, and reflected by the mirror 41. If the light source device 31 has only one laser diode 51, then the laser beam 33 emitted from the laser diode 51 is the parallel light 21. Again, the parallel light 21 is incident on the reflection mechanism 32, and reflected by the mirror 41 as collimated light.

The control device 13 controls the driver 42 of the reflection mechanism 32 such that the irradiation spot 24 travels along the reflection route 23 in FIGS. 7 and 8. The rotational speed of the mirror 41 is controlled according to the above process, resulting in the irradiation spot 24 travelling across the light incidence region 22 at a constant speed. When the irradiation spot 24 reaches the endpoint 23B (see FIG. 10) of the light incidence region 22, the control device 13 causes the light source device 31 to stop emitting the laser beam 33. After the mirror rotates to the point where the irradiation spot 24 (the optical axis of the irradiation spot 24) would pass the inversion point 23A and reaches the endpoint 23C, the control device 13 controls causes the light source device 31 to resume emitting the laser beam 33. As a result, as illustrated in FIG. 11, the amount of irradiating parallel light that reaches the light incidence region is smoothed from the endpoint P1 to the end point P2 in the light incidence region 22, and the photon flux density is smoothed in the whole light incidence region 22.

By way of example, in the photosynthetic microorganism culture apparatus 10, irradiation of the culture solution 20 is started at 6 o'clock in the morning, and ended at 6 o'clock in the evening. When sufficient microorganism density is reached, the photosynthetic microorganism is harvested by a method suitable for that photosynthetic microorganism.

### A Possible Modification

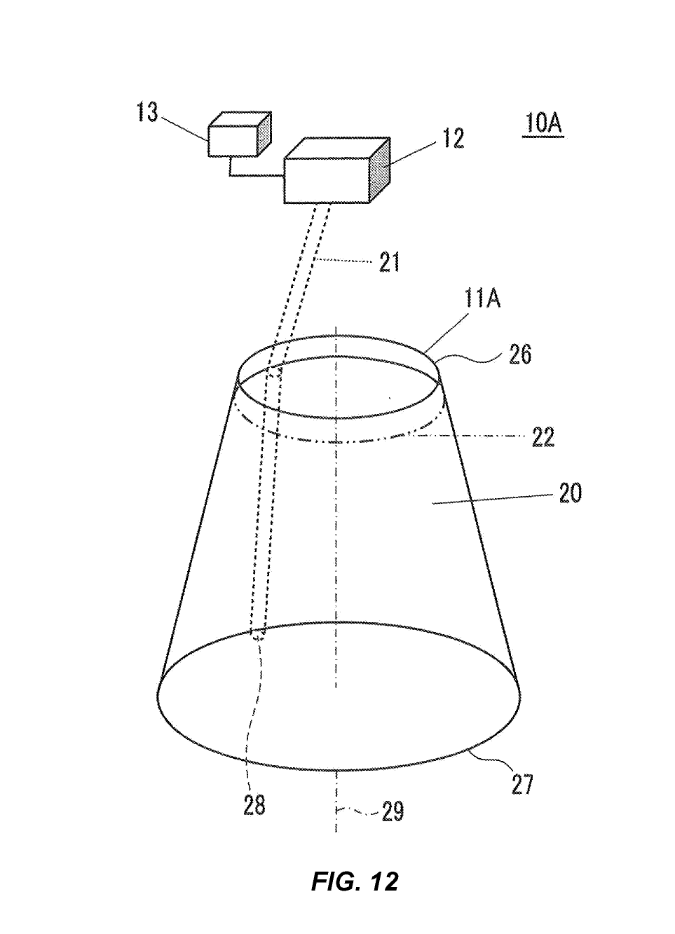

In a photosynthetic microorganism culture apparatus 10A of FIG. 12, a fermenter 11A has a circular opening 26 in its top. The liquid level of the culture solution is used as the light incidence region 22. Conceptually, the profile of the light incidence region 22 is matched with the profile at the liquid level of the culture solution 20. In this example, these profiles are circular. The bottom 27 of the fermenter 11A also has a circular shape. When the fermenter 11A is viewed from above, the center of the bottom 27 of the fermenter is aligned with the centers of the opening 26 and of light incidence region 22.

As illustrated in FIG. 12, the bottom 27 of the illustrated fermenter has a circular shape. The shape of the bottom 27 is set to account for refraction of the parallel light 21 incident on a light incident region 22 at or near the outside edge of the light incident region. The parallel light 21 incident on the region at or near the outer boundary edge of the light incidence region 22 is refracted at the liquid level, and reaches an irradiation point 28 at the bottom 27 of the fermenter. The shape of the bottom 27 of the fermenter is set such that outer edge of the bottom 27 of the fermenter is disposed adjacent to the irradiation point 28. In this case, the fermenter 11A has a truncated cone shape. A culture amount of the photosynthetic microorganism per unit area of the culture solution 20 can be maximized when the shape of the fermenter 11A is set by this method. When this method is applied to a square light incidence region 22, the shape of the fermenter is a truncated pyramidal shape.

What is claimed is:

1. A photosynthetic microorganism culture apparatus comprising:
a fermenter in which a culture solution that includes a photosynthetic microorganism is stored, the fermenter having a light incidence region through which light enters the culture solution that is being formed in the fermenter;
at least one light source that emits collimated light;
a reflection mechanism that reflects the collimated light in a predetermined direction as reflected collimated light; and
a controller that causes the reflected collimated light to travel periodically in a previously-fixed direction while irradiating the light incidence region with collimated light.

2. The photosynthetic microorganism culture apparatus according to claim 1, wherein the light source includes a laser diode.

3. The photosynthetic microorganism culture apparatus according to claim 1, wherein the controller controls the light source such that the culture solution is intermittently irradiated with the collimated light at a frequency corresponding to the time necessary for a photosynthetic reaction to occur in the photosynthetic microorganism.

4. The photosynthetic microorganism culture apparatus according to claim 1, wherein the controller controls the reflection mechanism such that the collimated light is reflected in a period corresponding to the time necessary for a photosynthetic reaction to occur in the photosynthetic microorganism.

5. The photosynthetic microorganism culture apparatus according to claim 1, wherein the controller controls the reflection mechanism to form a reflection route to moves from inside the light incidence region to outside the light incidence region and back again, stops the emission of collimated light when the reflection route moves outside light incidence region, and causes the light source to resume emitting collimated light when the reflection route returns to inside the light incidence region.

6. The photosynthetic microorganism culture apparatus according to claim 1, wherein the reflection mechanism includes:
a mirror in which a reflection angle is variable, the mirror being provided to be able to reflect the collimated light; and
a driver that changes the reflection angle of the mirror.

7. The photosynthetic microorganism culture apparatus according to claim 1, wherein the fermenter includes the light incidence region at a top thereof, and a bottom profile that corresponds with the position at which collimated light from the light source that is reflected toward the perimeter of the light incident region refracts to the bottom of the fermenter.

8. The photosynthetic microorganism culture apparatus according to claim 2, wherein the controller controls the reflection mechanism such that a reflection route is formed, the reflected collimated light returns to inside the light incidence region while moving along the reflection route from inside to outside of the light incidence region, the controller controls the collimated light source to stop emission of the emitted parallel light when the reflected collimated light moves from inside to outside the light incidence region, and to start emission of the collimated light again when an optical axis of the reflected collimated light moves from outside to inside the light incidence region if the cycle of irradiation is not complete.

9. The photosynthetic microorganism culture apparatus according to claim 3, wherein the controller controls the reflection mechanism such that a reflection route is formed, the reflected collimated light returns to inside the light incidence region while moving along the reflection route from inside to outside of the light incidence region, the controller controls the light source to stop emission of the collimated light when the reflected collimated light moves from inside to outside the light incidence region, and to start emission of the collimated light again when an optical axis of the reflected collimated light moves from outside to inside the light incidence region if the cycle of irradiation is not complete.

10. The photosynthetic microorganism culture apparatus according to claim 4, wherein the controller controls the reflection mechanism such that a reflection route is formed, the reflected collimated light returns to inside the light incidence region while moving along the reflection route from inside to outside of the light incidence region, the controller controls the light source to stop emission of the collimated light when the reflected collimated light moves from inside to outside the light incidence region, and to start emission of the collimated light again when an optical axis of the reflected collimated light moves from outside to inside the light incidence region if the cycle of irradiation is not complete.

11. The photosynthetic microorganism culture apparatus according to claim 6, wherein the controller controls the reflection mechanism such that a reflection route is formed, the reflected collimated light returns to inside the light incidence region while moving along the reflection route from inside to outside of the light incidence region, the controller controls the light source to stop emission of the collimated light when the reflected collimated light moves from inside to outside the light incidence region, and to start emission of the collimated light again when an optical axis of the reflected collimated light moves from outside to inside the light incidence region if the cycle of irradiation is not complete.

12. The photosynthetic microorganism culture apparatus according to claim 7, wherein the controller controls the reflection mechanism such that a reflection route is formed, the reflected collimated light returns to inside the light incidence region while moving along the reflection route from inside to outside of the light incidence region, the controller controls the light source to stop emission of the collimated light when the reflected collimated light moves from inside to outside the light incidence region, and to start emission of the collimated light again when an optical axis of the reflected collimated light moves from outside to inside the light incidence region if the cycle of irradiation is not complete.

13. The photosynthetic microorganism culture apparatus according to claim 2, wherein the controller controls the light source such that the culture solution is intermittently irradiated with the reflected collimated light at a frequency corresponding to time necessary for a photosynthetic reaction to occur in the photosynthetic microorganism.

14. The photosynthetic microorganism culture apparatus according to claim 2, wherein the controller controls the reflection mechanism such that the reflected collimated light is reflected in a period corresponding to the time necessary for a photosynthetic reaction to occur in the photosynthetic microorganism.

15. The photosynthetic microorganism culture apparatus according to claim 2, wherein the reflection mechanism includes:
a mirror in which a reflection angle is variable, the mirror being provided to be able to reflect the collimated light; and
a driver that changes the reflection angle of the mirror.

16. The photosynthetic microorganism culture apparatus according to claim 15, wherein the controller controls the light source such that the culture solution is intermittently irradiated with the reflected collimated light at a frequency corresponding to time necessary for a photosynthetic reaction to occur in the photosynthetic microorganism.

17. The photosynthetic microorganism culture apparatus according to claim 15, wherein the controller controls the reflection mechanism such that the collimated light is reflected in a period corresponding to the time necessary for a photosynthetic reaction to occur in the photosynthetic microorganism.

18. The photosynthetic microorganism culture apparatus according to claim 15, wherein the controller controls the reflection mechanism such that a reflection route is formed, the reflected collimated light returns to inside the light incidence region while moving along the reflection route from inside to outside of the light incidence region, the controller controls the light source to stop emission of the collimated light when the reflected collimated light moves from inside to outside the light incidence region, and to start emission of the collimated light again when an optical axis of the reflected collimated light moves from outside to inside the light incidence region if the cycle of irradiation is not complete.

19. A lighting device that lights up a fermenter in which a culture solution including a photosynthetic microorganism is stored, a light incidence region through which light enters the culture solution being formed in the fermenter,
the lighting device comprising:
one or a plurality of light sources that emit collimated light;
a reflection mechanism that reflects the collimated light emitted from the light source in a predetermined direction as reflected collimated light; and
a controller that causes the reflected collimated light to travel periodically in a previously-fixed direction to irradiate the light incidence region with the reflected collimated light.

20. A method for culturing a photosynthetic microorganism using a fermenter in which a culture solution including a photosynthetic microorganism is stored, a light incidence region through which light enters the culture solution being formed in the fermenter,
the method comprising the steps of:
emitting collimated light from one or a plurality of light sources;
reflecting the collimated light in a predetermined direction as reflected collimated light using a reflection mechanism that can variably control a reflection direction; and
controlling the reflection mechanism such that the reflected collimated light travels periodically in a previously-fixed direction.
